(19) United States
(12) Patent Application Publication
(10) Pub. No.: US 2011/0112586 A1
(43) Pub. Date: May 12, 2011

(54) METHODS AND DEVICES FOR PORTAL FIXATION TO THE SPINE

(76) Inventors: Jeffrey Allen Guyer, Boston, MA (US); Thomas Purcell, Del Mar, CA (US); Nathan Meyer, Vista, CA (US); Ali Araghi, Phoenix, AZ (US); Javier Garcia-Bengochea, Jacksonville, FL (US); Carl Lauryssen, Beverly Hills, CA (US); Moran Lorio, Bristol, TN (US)

(21) Appl. No.: 12/943,210

(22) Filed: Nov. 10, 2010

Related U.S. Application Data
(60) Provisional application No. 61/260,362, filed on Nov. 11, 2009.

(51) Int. Cl. A61B 17/56

(52) U.S. Cl. 606/86 A; 606/86 R

(57) ABSTRACT

A method and device for attaching a curvilinear access device having a movable top and an expandable working portal to the spine, the attachment including a holding arm assembly, posterior tang, anterior tang and/or a portal fixation pin.

### CROSS-REFERENCE TO RELATED APPLICATIONS

This application claims priority to U.S. Provisional Application No. 0109PR, filed on Nov. 11, 2009 as U.S. Provisional Application No. 61/260,362.

### BACKGROUND OF THE INVENTION

1. Field of the Invention

This invention relates generally to the field of surgery. In particular, the present invention relates to the field of surgical access to the spine.

2. Background of the Invention

This invention relates generally to the field of devices, methodologies and systems involved in lumbar interbody fusion, wherein an interbody device or implant is positioned between adjacent vertebrae in order to stabilize or fuse the vertebrae. Modern surgical techniques for this are relatively minimally invasive, in that improved techniques, instrumentation and implant design allow the site to be prepared and the implant to be introduced through one or several small incisions in the patient.

Surgical procedures known as lumbar interbody fusion (LIF) have become common over the past ten years. Particular techniques are typically designated by the direction of approach relative to the spine—anterior (ALIF), posterior (PLIF), transverse (TLIF), and extreme lateral (XLIF).

Many surgical procedures require the use of an access portal or cannula to deliver the LIF to the desired location of the spine. The distal end of the portal is typically positioned proximate the desired spinal location of the procedure. It is important the portal stay in position relative to the spine during the surgical procedure, but sometimes the relationship between the portal and spine is disrupted and the portal moves. It would be desirable to develop devices and methods to secure or couple an access portal to the spine during a surgical procedure.

### SUMMARY OF THE INVENTION

In a first aspect, embodiments of the present invention provide a device for attaching a posterior tang to a curvilinear access device having a movable top and an expandable working portal, the device includes a posterior tang guide configured to engage a posterior tang in a first position and a handle configured to move the posterior tang from the first position to a second position, wherein in the second position the posterior tang guide is configured to disengage with the posterior tang.

In another aspect, embodiments of the present invention provide a system for attaching a curvilinear access device at a surgical site of the spine of a patient, the system including posterior and anterior tangs extending from a distal end of the curvilinear access device configured to engage the spine, a holding arm assembly coupled to a proximal end of the curvilinear access device configured to couple the curvilinear access device, and a portal fixation pin configured to fit within one or more cannulas within movable tops of the curvilinear access device and extend from the distal end and engage the spine.

In another aspect, embodiments of the present invention provide a method for locking a curvilinear access device at a surgical site of the spine of a patient, the method including advancing a distal end of the curvilinear access device to the surgical site, coupling a holding arm assembly to a proximal end of the curvilinear access device, extending an anterior tang from a distal end of the curvilinear access device and engaging the spine, opening movable tops of the curvilinear access device forming a working portal, inserting a posterior tang guide having a detachable posterior tang through the working portal, extending a posterior tang from the distal end of the curvilinear access device by actuating the posterior tang guide to couple the posterior tang to the curvilinear access device and engage the spine, and removing the posterior tang guide from the curvilinear access device.

Further features and advantages of the invention, as well as structure and operation of various embodiments of the invention, are disclosed in detail below with references to the accompanying drawings.

### BRIEF DESCRIPTION OF THE DRAWINGS

The present invention is described with reference to the accompanying drawings. In the drawings, like reference numbers indicate identical or functionally similar elements.

FIG. 1 shows a patient in a prone position for performance of a posterior-lateral surgical procedure.

FIGS. 2A and 2B shows a curved access portal for use in the posterior-lateral surgical procedure in the closed and open configurations.

FIG. 3 shows the curved portal delivered over one or more dilators.

FIG. 4 shows the curved portal attached to a holding arm assembly.

FIG. 5 shows an implant inserted through the curved portal and implanted in the spine.

FIGS. 6A and 6B shows the curved portal and distal end in the deployed configuration including posterior and anterior tangs or tabs for attachment to the spine.

FIG. 7 shows a posterior tang guide.

FIG. 8 shows the loading of a posterior tang into the posterior tang guide.

FIGS. 9A-9D show the operation of the posterior tang guide deploying the posterior tang.

FIGS. 10A and 10B shows the posterior tang guide placed within the curved portal.

FIG. 11 shows a portal fixation pin

FIGS. 12A and 12B show the portal fixation pin with the curved portal.

### DETAILED DESCRIPTION OF THE INVENTION

Embodiments disclosed herein are merely exemplary of the invention. Specific structural and functional details disclosed herein are not to be interpreted as limiting, but merely as a basis for the claims and as a representative basis for teaching one skilled in the art to variously employ the present invention in virtually any appropriately detailed structure.

Many of the embodiments disclosed herein are disclosed with curvilinear spinal access methods and devices using in a posterior-lateral approach to the spine, such as the guided lumbar interbody fusion (GLIF) using the curvilinear or Arc portal disclosed in co-pending U.S. patent application Ser. Nos. 12/460,795 and 12/069,721, the entire disclosures of these applications are incorporated by reference. Embodiments of the present invention should not be limited curvilinear access methods and devices, and also should not be limited to a posterior-lateral approach. Embodiments of the present invention may also be used in many other surgical approaches to the spine, such as anterior (ALIF), posterior (PLIF), transverse (TLIF), and extreme lateral (XLIF). Embodiments of the present invention should also not be limited to the spine and may be used in other orientations and other surgical sites within the body.

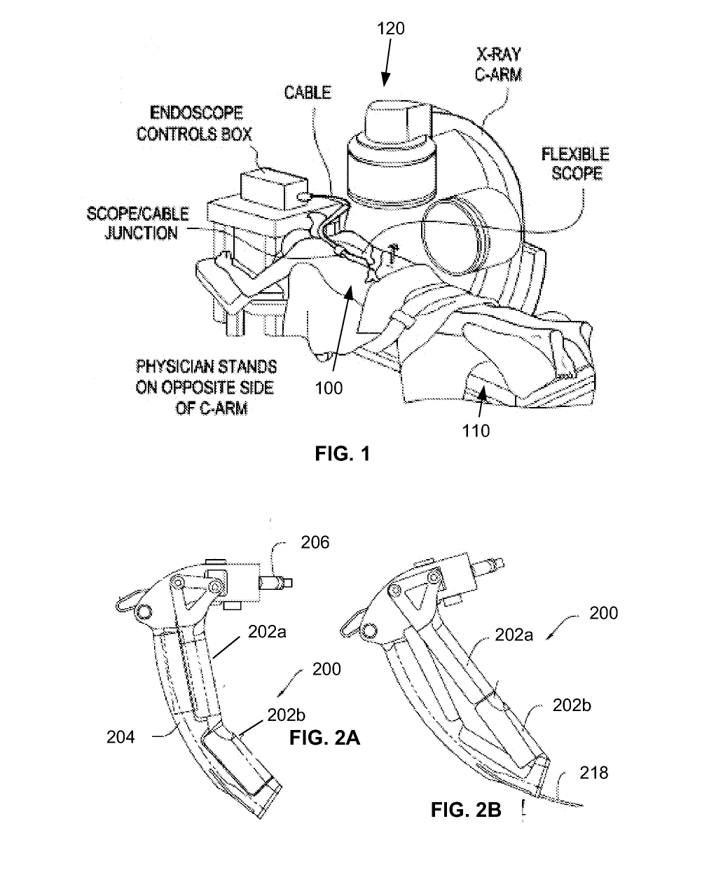

A guided lumbar interbody fusion (GLIF) procedure begins with placing a patient 100 in a prone position on the surgical table 110 and, with the aid of lateral fluoroscopy 120, adjusting the patient so that the operative disc space is generally perpendicular with the operating room floor, shown in FIG. 1. The GLIF technique accesses the lateral anterior spine through a curvilinear portal, the Arc Portal, while the patient is in the prone position for the purposes of implanting a device. This access and patient orientation offers many advantages over conventional approaches including allowing a larger graft implantation, eliminating the need to reposition a patient for posterior stabilization, persevering natural posterior stabilization elements, etc.

FIGS. 2A and 2B show a curved access portal 200 having a moveable top 202a, 202b. Examples of curved portal devices and procedures are disclosed in U.S. patent application Ser. No. 12/460,795. The curved portal 200 allows curved access to the spine while the patient is in the prone position having the following advantages over traditional lumbar interbody fusion: adding posterior fixation without rotating patient, minimizing nerve compression against TP compared to a straight oblique approach, delivering an implant with better anatomic physiology without requiring drastic repositioning (like TLIF req.), protecting anterior aspect, preserving posterior elements and protecting the bowels from injury. In the expanded or open configuration, the movable top allows direct visualization through the working portal, allowing the surgeons to confirm anatomy and ensure soft tissue is protected. Once the curved portal has been inserted into the patient and coupled to the desired area of the spine, any one of many surgical procedures can now be performed through the portal, including removal of annulus material, vertebral distraction, implant insertion, fusion procedures. Tools used in these procedures may include a rotating actuator, shaver blade, osteotomes, cobbs.

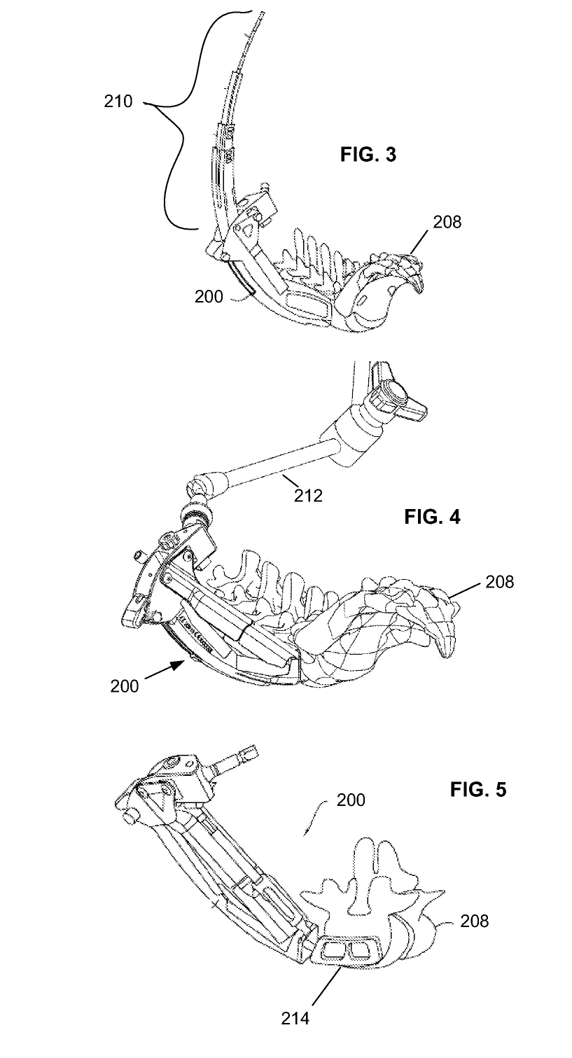

The curved portal 200 includes the moveable top 202a, 202b, portal slide 204, and fixation arm attachment 206. The curved portal 200 is delivered to the patient's lateral spine 208 area in the closed configuration, FIG. 2A and then the movable top is expanded or opened to provide direct visualization to the surgical site, FIG. 2B. The curved portal 200 is delivered over one or more dilators 210, shown in FIG. 3 and then can be opened in-situ after the dilators 210 are removed. Any number of dilators may be used. In some embodiments, the curved portal 200 can be supported using a holding arm assembly 212 attached to the fixation arm attachment 206, shown in FIG. 4. The other end of the holding arm assembly 212 is typically attached to the surgical table or other solid support. Once in position, the surgical procedure may be done and an implant 214 may be inserted through the curved portal 200 to the desired location, shown in FIG. 5.

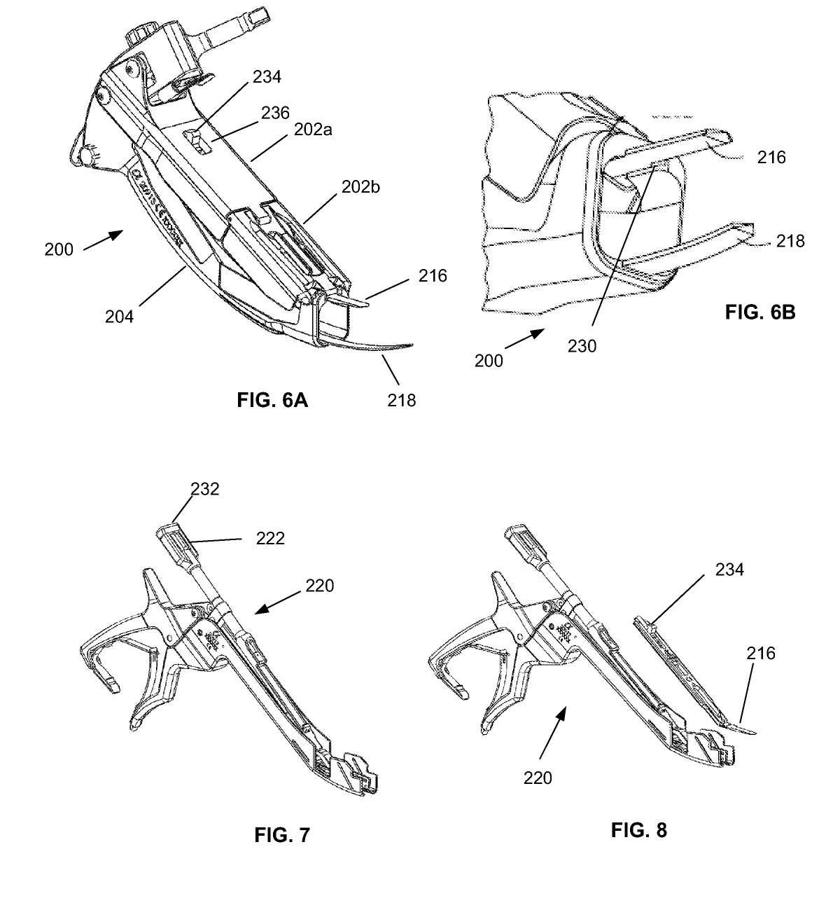

FIGS. 6A and 6B shows the curved portal 200 in the deployed configuration including posterior 216 and anterior 218 tangs or tabs for attachment to the spine 208. The posterior tang 216 fits within a distal attachment feature 230 of the curved portal 200. The anterior tang 218 may be movably coupled to the slide portal 204.

When the distal end of the curved portal 200 is in position at the surgical site and first opened, soft tissue typically obscures the surgeon's view of the operative site. This soft tissue needs to be identified, isolated and retracted out of the working channel of the arc portal. Previously instruments similar to elevators and penfields were used to accomplish this task; however the cantilevered forces innate to these instruments could not provide the mechanical advantage necessary to retract the soft tissue. Furthermore the free-hand nature of these tools made integrating with the connection features in the arc portal very difficult.

A posterior tang guide 220, shown in FIG. 7, is used with the GLIF technique to safely and repeatedly deliver and attach the posterior tang 216 to the curved portal 200 and move the tissue material away from the surgical site. FIG. 8 shows the loading of the posterior tang 216 into the posterior tang guide 220. A knob 222 is turned to constrain the posterior tang 216 in the posterior tang guide 220. The posterior tang guide 220 provides the necessary mechanical forces to the posterior tang 216 to sweep the portal clear of tissue, hold back nerve roots and tissue, and allow the posterior tang to properly integrate with the connection features in the arc portal. The posterior tang guide 220 allows the delivery of the posterior tang 216 in a safe and repeatable manner. This instrument transforms a previously cumbersome task into an easily automated procedure. The mechanisms allow tactile feedback to allow surgeons to better manipulate the soft tissue anatomy.

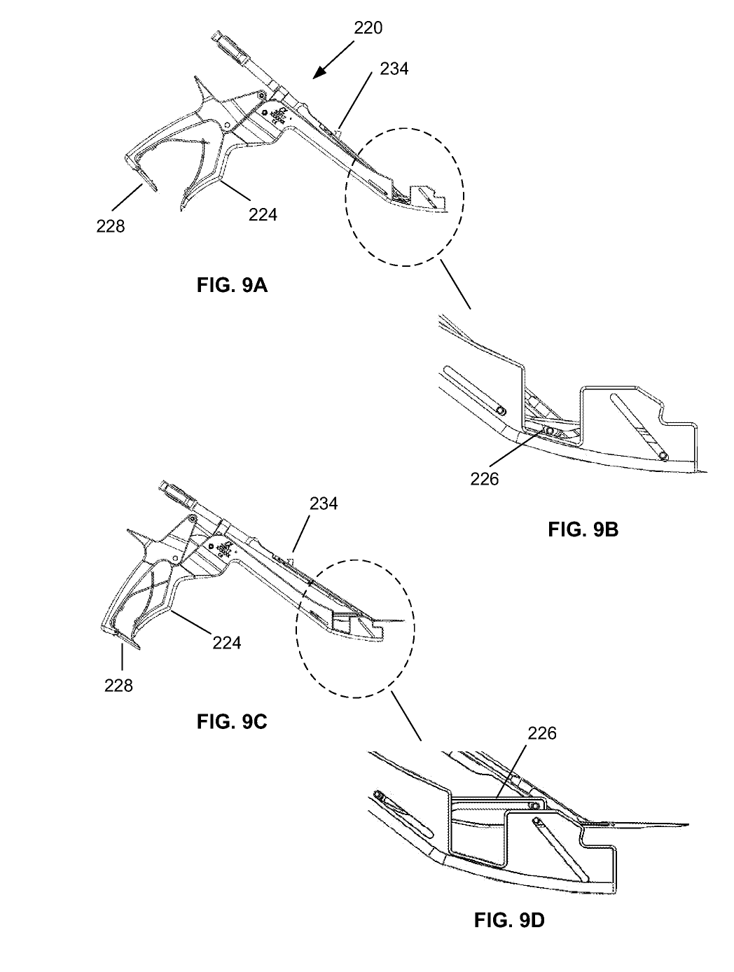

FIGS. 9A-9D show the operation of the posterior tang guide 220 deploying or lifting the posterior tang 216 (curved portal 200 not shown). Actuating a handle grip 224 of the posterior tang guide 220 raises a lifter 226 to move tissue posteriorly. When the handle 224 is squeezed enough a ratcheting mechanism 228 is engaged.

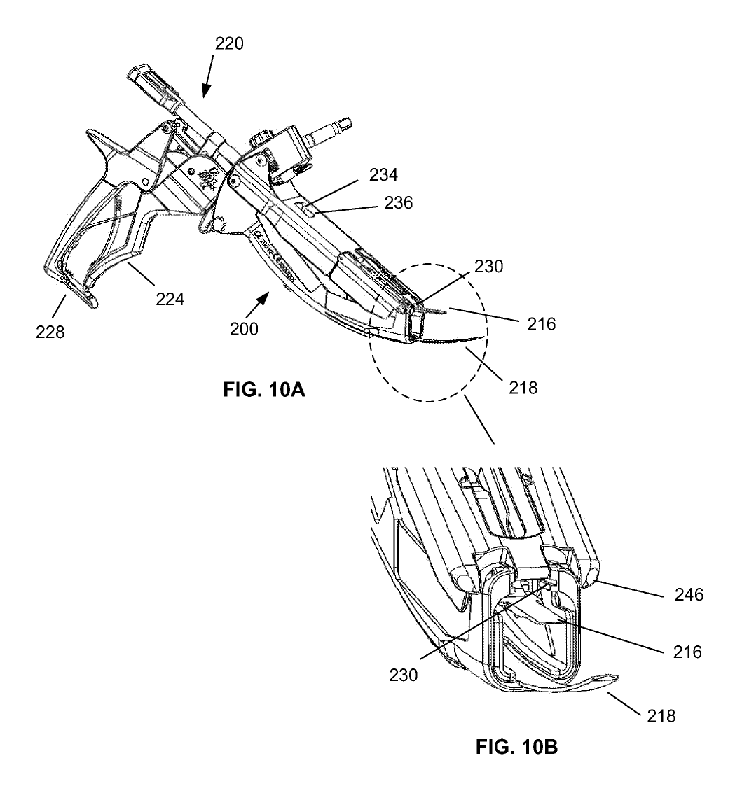

FIGS. 10A and 10B show the posterior tang guide 220 placed within the curved portal 200 and deploying the posterior tang 216. The handle 224 is squeezed and released to create a scraping motion of the posterior tang 216 along the lateral spine to move tissue away from the working portal. When the handle 224 is squeezed enough for the ratcheting mechanism 228 to engage, the lifter 228 moves the posterior tang 216 into engagement with the distal 230 and proximal 236 attachment features in the curved portal 200. The distal attachment may include distal wings on the posterior tang 216 that engage slots 230 in the curved portal 200. As the posterior tang 216 is advanced, a springed mechanism 234 enables the posterior tang 216 catch mechanism to integrate with the proximal window 236 in the movable top 202a of the curved portal 200. At this point the lifter 226 exposes the remainder of the confined channel allowing the posterior tang 216 to advance by impacting a strike plate 232. Once the posterior tang 216 is attached to the distal 230 and proximal 236 attachment features and coupled to the spine, the instrument 220 is removed, leaving the posterior tang 216. As shown in FIG. 6A, the curved portal 200 now has two tabs, posterior tang 216 and anterior tang 218, protruding from the distal end of the curved portal 200 that attach to the spine 208 during the procedure.

Once the working portal of curved portal 200 is in the open configuration, the surgeon may now perform the desired procedure which may include the implantation of an implant 214, or other suitable implants. Throughout the procedure proper stabilization of the curved portal 200 is imperative to a successful surgery, ensuring the impaction forces do not dislodge or move the instrument from its position against the lateral spine 208. In some surgeries, additional attachment means may be desired to hold the curved portal 200 to the spine 208.

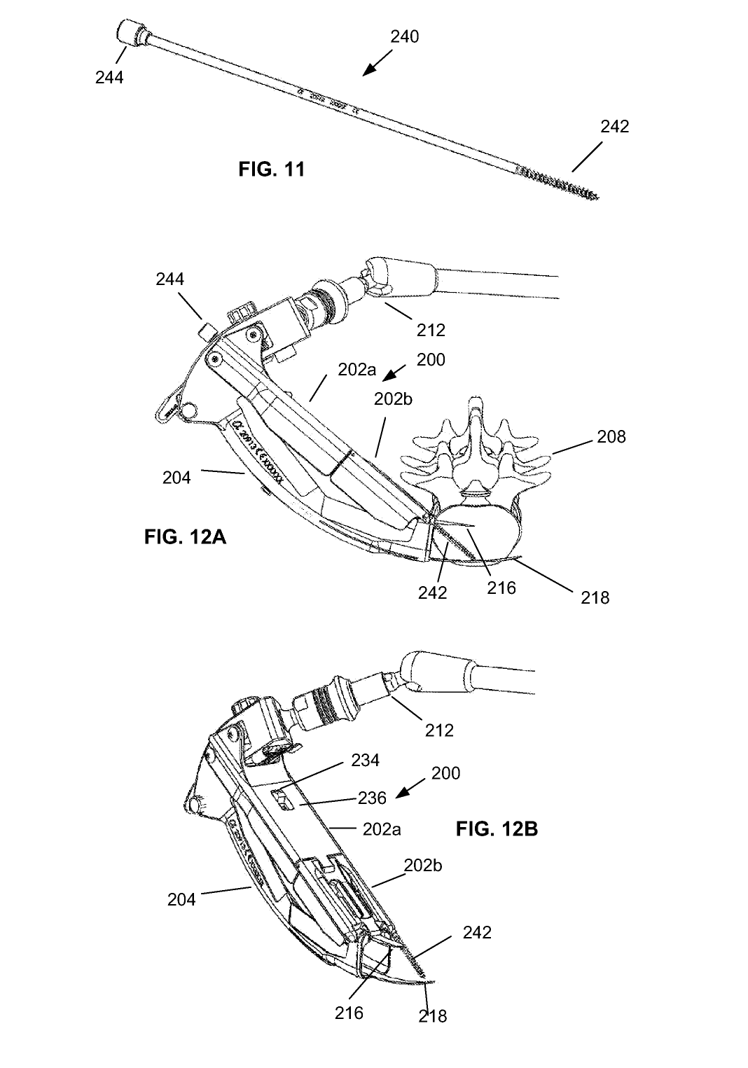

FIG. 11 shows a portal fixation pin 240 and FIGS. 12A and 12B show the portal fixation pin with the curved portal 200. The portal fixation pin 240 has a bone thread 242 on a distal end and a knurled barrel 244 with a hex features to drive the instrument into bone on a proximal end. To integrate the portal fixation pin 240 into the curved portal 200, one or more cannulas 246 are added to the moveable tops 202a, 202b of curved portal 200 through which the portal fixation pin 240 is delivered when the tops 202a, 202b are in the expanded or open configuration. The portal fixation pin 240 may also assist in keeping or locking the tops 202a, 202b in the expanded configuration. The portal fixation pin 240 allows the curved portal 200 to be mechanically joined to the anterior spinal column 208 and prevents both separation of the curved portal 200 from the spine 208 and anterior migration of the curved portal 200 from the surgical site.

The portal fixation pin 240 is delivered through cannula 246 located in the hinged moveable tops 202a, 202b of the curved portal 200. The portal fixation pin 240 is then driven into the vertebral body 208 to provide a mechanical fixation of the curved portal 200 against the spinal column. This fixation prevents curved portal 200 migration from the surgical site and prevents separation of the curved portal 200 from the spine 208.

Portal 200 stabilization is achieved using the table fixation arm 212, posterior tang 216, anterior tang 218 and portal fixation pin 240. Portal fixation pin 240 is designed to protrude up to the anterior tang 218 to provide maximum bone purchase in the adjacent vertebral bodies. Trajectory of portal fixation pin 240 is designed to prevent downward forces by creating a “tent staking” orientation where the portal fixation pin is the stake and to prevent pulling away from the spine by creating a wedge design.

In some embodiments, where the present invention's device and instrumentation are used to attach an access portal to the spine for implant delivery, the implants may include, but are not limited to: bone screws, plates, interbody devices, artificial discs, or any other implants. Further, the present invention's device and methodology can be used in any number of surgical procedures, including nucleus replacement, total disc replacement, interbody fusion, discectomy, neural decompression, implant delivery (whether for fixation purposes and/or stabilization), or any other procedure.

Example embodiments of the methods and components of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments, but should be defined only in accordance with the following claims and their equivalents.

What is claimed:

1. A device for attaching a posterior tang to a curvilinear access device having a movable top and an expandable working portal, comprising:
a posterior tang guide configured to engage a posterior tang in a first position; and
a handle configured to move the posterior tang from the first position to a second position, wherein in the second position the posterior tang guide is configured to disengage with the posterior tang.

2. The device of claim 1, wherein the posterior tang guide is configured to fit within an expandable working portal of the curvilinear access device.

3. The device of claim 1, wherein the posterior tang guide includes a lifter mechanism coupled to the handle configured to move the posterior tang from the first position to the second position.

4. The device of claim 1, wherein the posterior tang guide disengages with the posterior tang once the posterior tang couples with one or more attachment features of the curvilinear access device.

5. The device of claim 1, further comprising a strike plate configured to receive an impact force to advance the posterior tang.

6. A system for attaching a curvilinear access device at a surgical site of the spine of a patient, comprising:
posterior and anterior tangs extending from a distal end of the curvilinear access device configured to engage the spine;
a holding arm assembly coupled to a proximal end of the curvilinear access device configured to couple the curvilinear access device; and
a portal fixation pin configured to fit within one or more cannulas within movable tops of the curvilinear access device and extend from the distal end and engage the spine.

7. The system of claim 6, wherein the posterior and anterior tangs extend distally in a first direction and the portal fixation pin extends distally in a second non-parallel direction.

8. The system of claim 6, wherein the portal fixation pin extends from the distal end to the anterior tang.

9. The system of claim 6, wherein the portal fixation pin extends at an angle relative to the anterior tang.

10. A method for locking a curvilinear access device at a surgical site of the spine of a patient, the method comprising:
advancing a distal end of the curvilinear access device to the surgical site;
coupling a holding arm assembly to a proximal end of the curvilinear access device;
extending an anterior tang from a distal end of the curvilinear access device and engaging the spine;
opening movable tops of the curvilinear access device forming a working portal;
inserting a posterior tang guide having a detachable posterior tang through the working portal;
extending a posterior tang from the distal end of the curvilinear access device by actuating the posterior tang guide to couple the posterior tang to the curvilinear access device and engage the spine; and
removing the posterior tang guide from the curvilinear access device.

11. The method of claim 10, further comprising engaging the spine with a portal fixation pin configured to fit within one or more cannulas within movable tops of the curvilinear access device in the open position.
